(19) United States
(12) Patent Application Publication
(10) Pub. No.: US 2009/0198152 A1
(43) Pub. Date: Aug. 6, 2009

(54) FINGER TIP TOURNIQUET

(76) Inventor: Stanley Kim, Upland, CA (US)

Correspondence Address: SHELDON MAK ROSE & ANDERSON PC, 100 Corson Street, Third Floor, PASADENA, CA, 91103-3842, US

(21) Appl. No.: 12/364,387

(22) Filed: Feb. 2, 2009

Related U.S. Application Data
(60) Provisional application No. 61/063,249, filed on Feb. 2, 2008.

(51) Int. Cl. A61B 17/00; A61B 5/151; A61B 5/15

(52) U.S. Cl. 600/583; 606/201; 600/573

(57) ABSTRACT

A tourniquet comprising a pair of compression pads and a spring which is applied to a finger tip to selectively restrict venous blood outflow from the finger tip without compromising arterial blood influx.

The present application claims the benefit of priority from copending Provisional Patent Application No. 61/063,249, filed Feb. 2, 2008 and titled “A Safe Finger Tip Tourniquet,” the contents of which are hereby incorporated by reference in their entirety.

### FIELD OF INVENTION

The present invention relates to a tourniquet applied to a finger tip to selectively restrict venous blood outflow from the finger tip without compromising arterial blood influx. This tourniquet provides a very safe method to increases blood pooled in the finger tip, making it easier to obtain a sufficient amount of blood for various laboratory tests.

### BACKGROUND

Tourniquets are used to restrict a blood supply to a body part distal to the tourniquet application. By occluding the blood supply to an extremity completely, bleeding from the injured area will cease.

Another common application of tourniquets is to help to withdraw blood from or inject drugs into veins. While hard pressure occludes both arteries and veins, shutting off all blood supplies completely, moderate or mild pressure can occlude venous circulation only because of the higher arterial blood pressure compared to venous blood pressure. By applying compression pressure only to block the venous blood draining, the veins distal to the tourniquet area will bulge out, so as to make it easy for medical staff to phlebotomize from or inject medicine into the veins. Similarly, by tightening the finger tip with a rubber band or a finger tourniquet as disclosed by Talen et al. in U.S. Pat. No. 5,893,870, the blood circulation in the finger tip can be restricted to help obtain more blood by a finger stick puncture.

However, such methods often block both arterial and venous blood circulation completely, which can cause discomfort, serious ischemia or irreversible tissue injury unless used very briefly. In addition, medical journals have reported tragic accidents requiring finger amputation by a forgotten finger tourniquet.

### SUMMARY

The present invention comprises a safe finger tourniquet and a method of using such a tourniquet for a finger stick laboratory test. The finger tourniquet in this present invention restricts mainly venous blood outflow only from the finger tip, not compromising more important arterial blood circulation. Therefore, this tourniquet effectively keeps more blood in the finger tip, thereby increasing patients' comfort without the risk of serious finger ischemia even with a prolonged application.

### DRAWINGS

FIG. 1 is a view of the ventral (lower, palm) side of the distal end of a finger showing both digital arteries (black in color) and veins (white in color).

FIG. 2 is a view of the dorsal (upper, knuckle) side of the finger illustrated in FIG. 1 showing both digital arteries (black in color) and veins (white in color).

FIG. 3 is a cross sectional view of the finger illustrated in FIG. 1 at the level of the middle phalanx, showing arteries (thick black circle), veins (white thin circle), digital bone, and ligaments.

FIG. 4 is a cross sectional view of the middle phalanx of the finger illustrated in FIG. 1 with the present finger tip tourniquet applied, showing compressed digital veins and the patent digital arteries.

FIG. 5 is a perspective view of the ventral side of a finger showing the tourniquet of FIG. 8 applied to the finger and having a lancing device with a lancet inside ready to puncture the finger tip.

FIG. 6 is a perspective view of the dorsal side of a finger showing the tourniquet of FIG. 8 applied to the finger.

FIG. 7-A is a perspective view of the present finger tip tourniquet with the compression pads detached.

FIG. 7-B is the perspective view of the compression pad.

FIG. 8 is a perspective view of the present finger tip tourniquet.

FIG. 9 is a perspective view of another embodiment of the present finger tip tourniquet.

FIG. 10 is a perspective view of a further embodiment of the present fingertip tourniquet.

FIG. 11 is a perspective view of yet another embodiment of the present finger tip tourniquet with a leaf spring placed toward the handle.

FIG. 12 is a perspective view of the finger tip tourniquet of FIG. 11, with the leaf spring moved toward the compression pad.

FIG. 13 is a perspective view of the finger tip tourniquet of FIG. 11 and FIG. 12 with the compression pads opened.

FIG. 14 is a perspective view of a tension pressure level adjustment system for the finger tip tourniquet of FIG. 11 and FIG. 12 in which the end of the leaf spring is placed in a longitudinal groove of the handle toward the compression pad.

FIG. 15A is a perspective view of an additional embodiment of the present tourniquet.

FIG. 15B is a side perspective view of the tourniquet of FIG. 15A worn on a finger.

FIG. 15C is a top view of the tourniquet of FIG. 15A.

FIG. 15D is a top view of the tourniquet of FIG. 15A worn on a finger.

FIG. 16 is a perspective view of the side of a finger showing the tourniquet of FIG. 8 applied to the finger in a vertical position.

FIG. 17A is a perspective view of another embodiment of the present tourniquet.

FIG. 17B is a side perspective view of the tourniquet of FIG. 17A worn on a finger.

FIG. 17C is a ventral perspective view of the tourniquet of FIG. 17A worn on a finger.

FIG. 17D is a dorsal perspective view of the tourniquet of FIG. 17A worn on a finger.

FIG. 18 illustrates the use of the tourniquet of FIG. 15A.

FIG. 19A is a side view illustrating a further embodiment of the present tourniquet on a finger, positioned for obtaining a blood sample.

FIG. 19B is a side perspective view of the tourniquet and finger shown in FIG. 19A.

### DESCRIPTION

### Definitions

As used herein, the following terms and variations thereof have the meanings given below, unless a different meaning is clearly intended by the context in which such term is used.

“Finger tip” refers to the distal portion of the finger of a subject, in particular a human subject. The finger tip is located in the distal phalanx of the finger. “Leaf spring” refers to a flat spring, i.e. a sheet of flat material that returns to its shape or position after being pushed, pulled, or pressed, and which thus can be elastically deformed. Leaf springs are typically bent.

“Smooth” describes a surface which is free from projections or unevenness.

“Spring” refers to an elastic device that returns to its shape or position after being tensioned (placed under tension), such as by pushing, pulling, or pressing the spring.

As used herein, the term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

### Finger Circulation

The present invention takes advantage of a unique blood circulation system in the finger 12 as illustrated in the present drawings. Blood circulation consists of 2 systems, arterial blood influx and venous blood draining. In between the arterial and venous systems, a very small capillary vessels bridge the 2 systems. Diabetic patients puncture the finger tips 14 to obtain capillary blood for a blood sugar test.

The digital arteries branch out from the larger Ulnar and Radial arteries of the arm and supply blood to the finger 12. The digital arteries run from the level of the finger webs on each lateral side of the fingers 12 all the way up to the finger tips 14, and the draining digital veins run on both ventral side 16 and dorsal side 18 of the fingers 12.

By compressing the ventral side 16 and dorsal side 18 of the fingers 12 but sparing the lateral (distal and medial) sides, only digital veins can be occluded while allowing the arteries patent. This will restrict the venous blood outflow only from the finger tips 14 without compromising more important arterial blood influx. As a result, the capillary blood pressure will increase temporarily in the present method until the blood is eventually drained out through detour collateral venous channels. Therefore, serious irreversible ischemic tissue injury can be avoided.

With the higher capillary blood pressure, a very tiny finger stick to the finger tip 22 will produce more blood volume, sufficient for laboratory tests. This means less pain due to the use of such a smaller and thinner lancet needle, faster puncture wound healing, and less waste of the test strips due to insufficient blood amount for the testing. In addition, patients will feel much more comfortable while this finger tourniquet is applied because the whole finger 12 is not wrapped tightly and completely, but partially. It is especially true when the compression pads of the tourniquet have soft cushion linings. Most importantly, this tourniquet method reduces or eliminates the risk of serious irreversible tissue necrosis from the prolonged application such as the forgotten finger tourniquet.

FIGS. 1 and 2 show the ventral (lower) side 16 and dorsal (upper) side 18 of the middle finger and its blood circulation system. The digital arteries 20, colored in black, run toward the finger tip 22 on both lateral sides of the finger 12. The digital arteries 20 supply arterial blood toward the finger tip 22. The digital veins 21, colored in white, however, run on the more ventral side 16 and dorsal side 18 of the finger 12. The digital veins 21 drain venous blood from the finger 12 toward the palm.

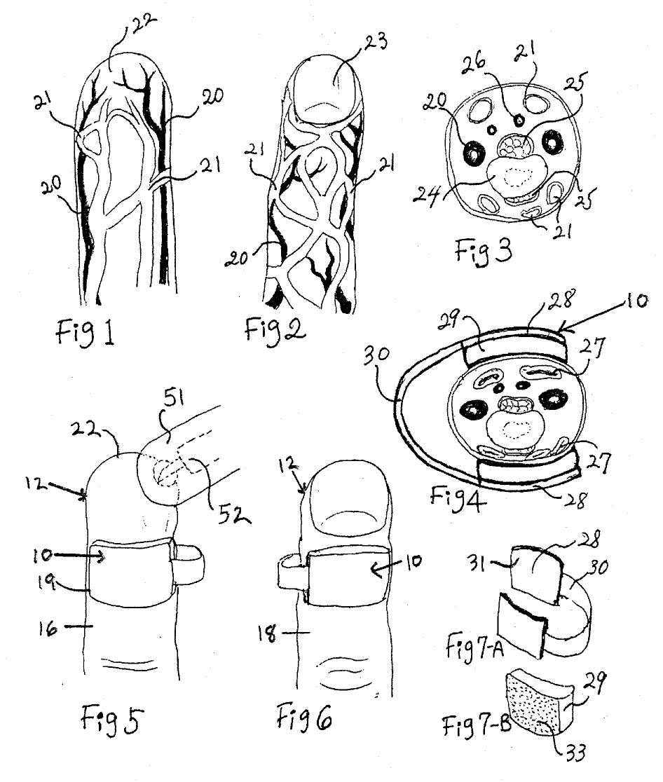

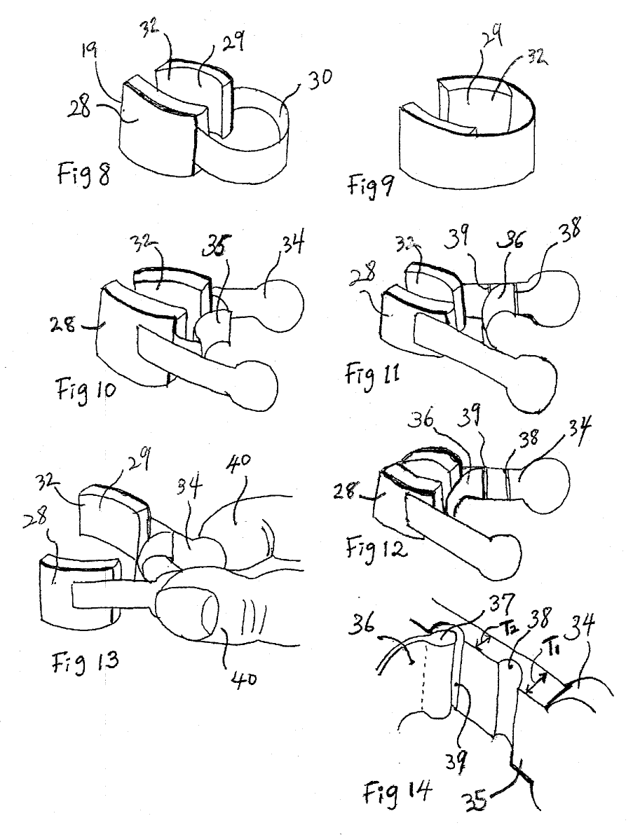

FIG. 3 is a cross sectional view of middle finger at the level of middle phalanx. The digital arteries 20 are located in both lateral side of the finger 12, and the digital veins 21 more in the ventral side 16 and dorsal side 18 of the finger 12. In the center is the phalangeal bone 24 on which the ligaments 25 are attached. There are small branches 26 of the digital arteries elsewhere in the finger 12 as well. It can be seen from these illustrations that by selectively compressing the ventral side 16 and dorsal side 18 of the finger 12, the venous blood outflow can be restricted while the arterial blood influx is allowed, which results in trapping more blood in the distal portion of the finger 12, i.e. the finger tip 22, and the capillary and venous pressure in the finger tip area will rise.

FIG. 4 shows a cross sectional view of the finger after the present finger tip tourniquet 10 is applied at the level of the middle phalanges. The pressure on both ventral side 16 and dorsal side 18 of the finger 12 by the tourniquet 10 results in the compressed digital vein 27 keeping more blood in the finger tip 22, and raising the capillary and venous blood pressures in the finger tip 22.

### Tourniquet

FIGS. 7-A and 7-B show the parts of one embodiment of the present finger tip tourniquet 10. FIG. 7-A shows the main frame of the finger tip tourniquet 10, consisting of the compression pad 28 and the tension (spring) connector 30. The compression pad 28 preferably has a mild curvature to easily fit the round finger. The tension connector 30 has a function of a leaf spring that resists outward stretching of the compression pad 28.

FIG. 7-B shows a cushion lining 29 for the compression pad 28. It is also preferably slightly curved to fit the curved compression pad 28. One side of the cushion lining 29 is treated with adhesives 33 in order to firmly attach onto the inner side 31 of the compression pad 28. The cushion lining 29 can be disposable as it may get dirty and stained with blood. The outer side 32 of the cushion lining 29 is preferably smooth both for comfort of the patient and in order to contact a sufficient surface area to act as a tourniquet. The materials for the cushion lining 33 also can be cotton, synthetic soft plastic rubber or sponge, or other materials.

FIG. 8 illustrates the finger tip tourniquet 10. The shape, size, configuration, materials of the finger tip tourniquet 10 can be of various origins as long as they serve the purpose of this invention. For example, if a patient wants a special personal finger tip tourniquet 10, the compression pad 28 and the tension connector 30 connect can be made of silver. On the other hand, one made of thermoplastic materials can be a very economical alternative. In an alternative embodiment, the compression pad 28 can be used without a cushion lining 29. In this embodiment, the compression pad 28 is preferably smooth, for comfort of the patient and to contact a sufficient surface area to act as a tourniquet.

FIG. 9 shows another embodiment of the finger tip tourniquet 10. The compression pad 28 and the tension connector 30 are constructed as one piece. This type may be more economical for a mass production.

FIG. 10 illustrates another embodiment of the finger tip tourniquet 10. A handle 34 is attached to the compression pad 28, and an M-shaped leaf spring 36 is used. When both handles 34 are pressed, the compression pads 28 will open to clip the finger 12. Instead of the M-shaped spring, a coil spring or other type of spring can also be used.

FIG. 11 shows another embodiment of the finger tip tourniquet 10 that has a tension pressure level adjustment system, illustrated in FIG. 14. The tension pressure onto a finger to which the tourniquet 10 is applied can be adjusted by moving the leaf spring 36 placed in between the handle 34. The tension pressure adjustment system of FIG. 14 consists of the longitudinal grooves 38, 39 on the inner wall 35 of the handle 34, the leaf spring 36 and a cylindrical column 37 provided on both ends of the leaf spring 36.

When the cylindrical column 37 are engaged in the longitudinal groove 38, which is nearer the handle in the embodiment shown in FIGS. 12 and 14, the tension of the leaf spring 36 decreases as does the compression pressure of the compression pad 28 onto the finger 12. When the leaf spring 36 is pushed inward to have the cylindrical column 37 engaged in the longitudinal groove 37 which is nearer the compression pad, the tension of the leaf spring 36 increases as does the compression pressure of the compression pad 28 onto the finger 12. The thickness of the wall of the handle between the longitudinal grooves (T2) is preferably thinner than that of the wall of the handle outside the grooves (T1). This feature helps to keep the leaf spring 36 from slipping out of the longitudinal grooves 38, 39.

In FIG. 11, the spring leaf 36 is engaged in the groove toward the handle 38. As a result, the compression pads 28 are not tightly closed due to less tension in the side of the compression pad 28. In FIG. 12, the spring leaf 36 is engaged in the groove toward the compression pad 39, and the compression pads 28 are more tightly closed due to increased tension. FIG. 13 shows the compression pads 28 open as the handles 34 are pinched by the fingers 40.

The tension connector 30 can be of various sizes, lengths, thicknesses, widths, shapes, colors and configurations, and can be made of metal, plastics, wood, or other tensile materials. The tension and pressure generated by the tension connector 30 can vary depending upon the size, thickness and configuration of the patient's finger as well as the patient's tolerance of certain compression pressures on the finger.

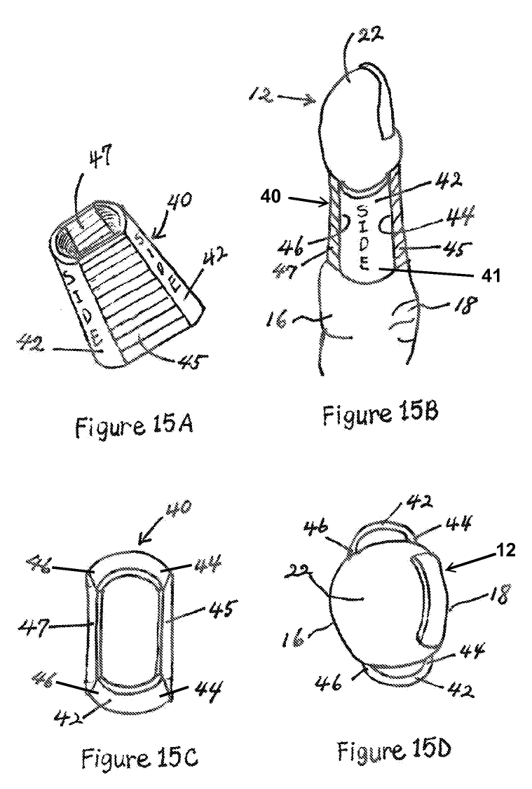

In another embodiment, shown in FIGS. 15A-15D, the present tourniquet is cylindrical and is placed on the finger 12 by placing the finger tip 22 through the cylindrical tourniquet 40. Cylindrical tourniquet 40 comprises two rigid compression pads 42, which can, for example, be made from a rigid material such as PVC or polycarbonate plastic. Each of the rigid compression pads 42 has a proximal side 44 and a distal side 46. In this embodiment, the proximal side 44 is proximate the dorsal surface 18 of the finger 12 when worn by a user, as shown in FIG. 15B, and the distal side 46 is proximate the ventral surface 16 of the finger 12. Preferably, the interior surfaces of the compression pads 42 (facing the surface of the finger 12 when worn by a user) are convex so as to avoid compressing the lateral sides of the finger where the arteries are running, thus allowing the influx of the arterial blood into the finger tip.

The proximal sides 46 of each of the rigid compression pads 42 are attached to and connected by a piece of elastic material 45. Likewise, the distal sides 44 of each of the rigid compression pads 42 are attached to and connected by a piece of elastic material 47, though in some embodiments the elastic material pieces 45 and 47 can comprise a continuous piece of elastic material.

The distance between the proximal side 44 and distal side 46, however, is less than the thickness of the finger 12 (i.e., the distance between the dorsal surface 18 and the ventral surface 16). The elastic materials 45, 47 are sized so that they must be stretched in order to be placed around a medial portion of a finger (such as around the medial phalanx), cylindrical tourniquet 40 thereby providing compression on the finger which is sufficient for the cylindrical tourniquet 40 to act as a tourniquet. The pressure exerted by the elastic materials 45, 47 is directed primarily to the ventral surface 16 and dorsal surface 18 of the finger 12.

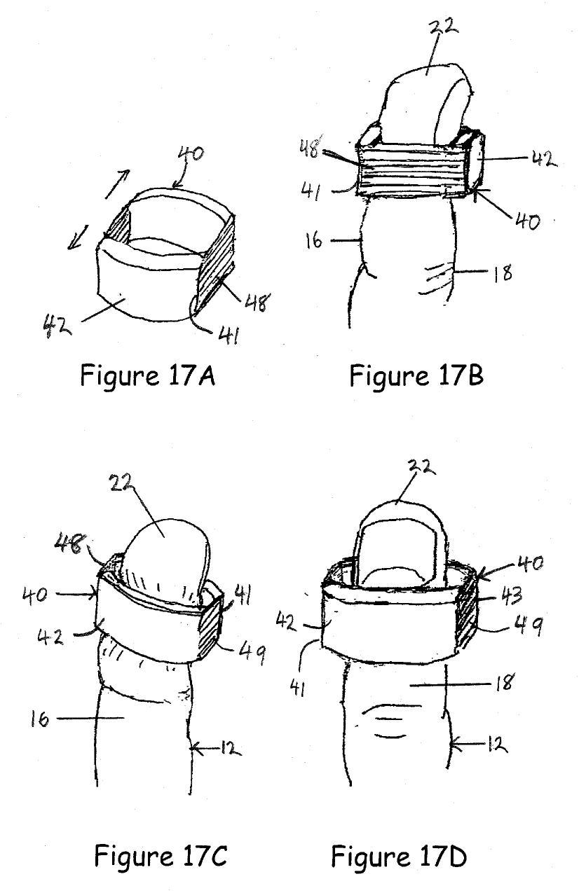

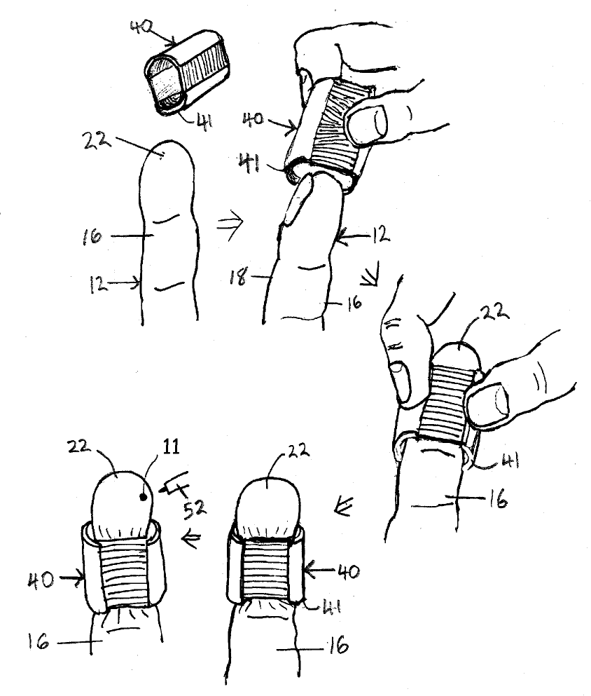

In an alternative embodiment, shown in FIGS. 17A-17D, the cylindrical tourniquet 40 is configured such that the distance between the medial side 41 (closer to the thumb) and lateral side 43 (closer to the pinky finger) of each of the rigid compression pads 42 is greater than the width of the finger 12, i.e. the distance along either the dorsal or ventral surface of the finger that is perpendicular to the longitudinal axis of the finger. Elastic material 48 attaches to and connects the respective medial sides of the compression pads 42, and elastic material 49 likewise attaches to and connects the lateral sides of compression pads 42.

As in the earlier described embodiment of the cylindrical tourniquet 40, the elastic materials (48, 49) are sized so that they must be stretched in order to be placed around a medial portion of a finger (such as around the medial phalanx). In this embodiment, however, pressure on the dorsal surface 18 and ventral surface 16 of the finger 12 is exerted by the rigid compression pads 42. The elastic materials (48, 49) in this case do not exert pressure directly on the surface of the finger 12, but act to draw the compression pads 42 toward each other and thereby provide compression.

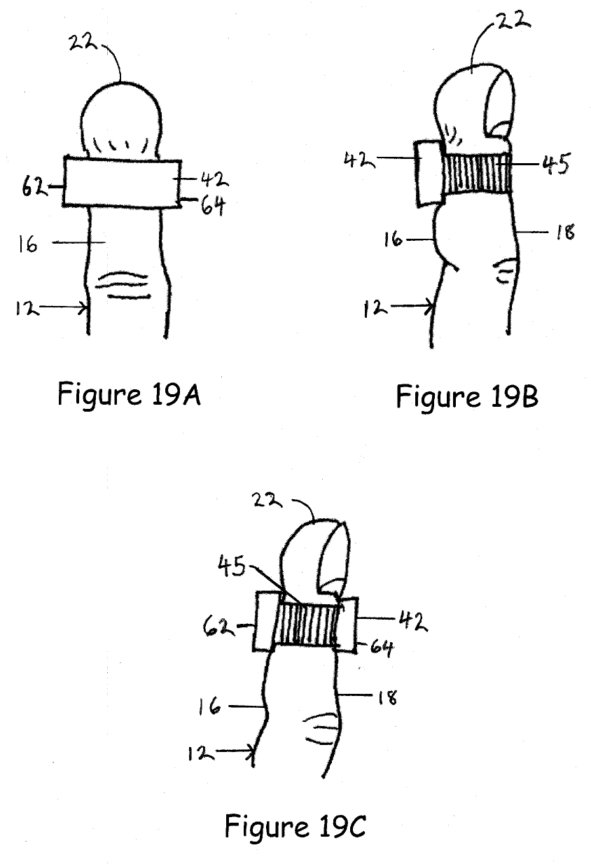

In a further embodiment, shown in FIGS. 19A and 19B, a tourniquet having only one compression pad 42 is used, together with an elastic material 45 connecting the medial sides 62, 64 of the compression pad 42. It has been found that compressing only the ventral side 16 of a finger 12 with a rigid compression pad 42 is sufficient to raise blood pressure at the distal end of the finger, for the purpose of obtaining a blood sample. In this embodiment, the distance between medial sides 62 and 64 in this embodiment is greater than the width of the finger 12, i.e. the distance between the two lateral sides of the finger. In this way, the compression pressure will not be applied on the lateral sides of the finger where the digital arteries run. FIGS. 19A and 19B illustrate the placement of the rigid compression pad 42 in this embodiment of the present tourniquet on the ventral surface 16 of the finger 12, while the elastic material 45 contacts the dorsal surface 18.

### Methods of Use

The present tourniquet 10 (including the cylindrical tourniquet embodiment) is placed on a finger proximal of the finger tip 22 in order to increase arterial blood pressure at the tip of the finger 12 and thus increase the flow of blood from a puncture wound at the proximal end of the finger 12. Preferably, the tourniquet 10 is placed between the middle and distal phalanx, just below the nail, in order to increase blood pressure in the distal phalanx, although it can also be placed in other positions, such as in a more proximal position around the middle phalanx of the finger.

Using the embodiments of the tourniquet shown in FIGS. 4-14, after tensioning the spring connector 30 so as to increase the distance between the outer sides 32 of the compression pads 28, the tip 19 of the compression pads can be moved laterally from one lateral side of a finger (such as in the medial phalanx) toward the other lateral side, thereby positioning one of the compression pads 28 over the dorsal surface 18 of the finger and the other compression pad 28 over the ventral surface 16 of the finger 12. A user then releases tension in the spring connector 30 and allows the compression pads to mover closer together until they contact the surface of the skin of the finger 12. The compression pads 28 then compress the dorsal 18 and ventral 16 surfaces of the finger in order to function as a tourniquet. Although it is desirable to apply this finger tourniquet 10 near the finger tip 14, it also can be placed anywhere in the finger depending on the patient's preference.

FIG. 5 shows the present tourniquet 10 applied to the ventral side 16 of the finger 12, which is being punctured by a lancet 52 inside the lancing device 51 at the tip 22 of the finger 12. Once blood emerges through the puncture wound, it can be collected for use, e.g., in a blood glucose test, or for any of a number of other diagnostic tests.

FIG. 6 shows the dorsal side 18 of the finger 12 with the finger tip tourniquet 10 applied. Because the finger tip 22 is congested with the trapped blood, a finger prick will produce more capillary bleeding even with a very small and thin lancet needle. Although the digital veins 21 are compressed, eventually the trapped blood will be drained toward the palm because of the collateral channels of the venous system. As the capillary and venous blood pressure in the finger tip 22 rise, small branches of the digital vein 21 in the not-compressed area of the finger will open to drain the trapped blood. It is like a congested highway situation when drivers start to use small detour roads after getting off the congested highway. Therefore, the finger tip 22 will not have severe ischemic damage from the prolonged use of the finger tip tourniquet 10 of the present invention.

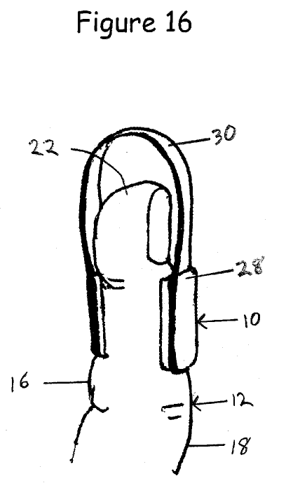

In an alternative procedure, the tourniquet 10 can be placed on a finger 12 by advancing the tip 19 of the compression pads from the tip 22 of the finger toward the proximal end of the finger. In this embodiment, once the tourniquet 10 is positioned on the finger 12, the spring connector 30 extends from one compression pad 28 past the tip 22 of the finger and then curves around the tip 22 of the finger to connect to the second compression pad 28. This is illustrated in FIG. 16.

The application of the cylindrical tourniquet of FIGS. 15A-15D is shown in FIG. 18. As shown in that figure, the proximal end 41 of the rigid compression pads 42 is first advanced past the tip 22 of the finger 12 such that the tourniquet 40 is placed around the finger, i.e. the interior surfaces of the rigid compression pads 42 face the skin of the finger 12. The tourniquet 40 is positioned such that the rigid compression pads contact the lateral sides of the finger 12, while the elastic material (45, 47) contacts the dorsal 18 and ventral 16 surfaces of the finger. Once placed on desired portion of the finger, the elastic material exerts pressure and increases blood pressure in the portion of the finger distal of the distal end of the tourniquet 40. The cylindrical tourniquet of FIGS. 17A-17D is applied in a like manner, except that the rigid compression pads 42 are placed in contact with the dorsal 18 and ventral 16 surfaces of the finger 12.

Another application of the finger tip tourniquet 10 is to reduce bleeding from a finger injury. By placing the compression pads 28 on the lateral sides of the finger instead on the ventral and dorsal sides, the digital arteries 20 are mainly compressed. Because of the presence of the small branches of the digital artery elsewhere other than the lateral sides of the finger, while it effectively controls the bleeding from the finger tip injury, this application will not cause complete occlusion of the arterial blood supply system to the finger tip 22, thus reducing the danger of completely shutting off the arterial circulation by the application of tourniquets of the prior art. This method of applying a finger tip tourniquet thus reduces bleeding from a finger injury substantially without the risk of serious ischemic tissue damage that can be caused by finger tourniquets of the prior art. This method of finger tip tourniquet application can also be useful for an elective finger surgery for the same reasons described above.

By contrast, in the prior art, when bleeding from the injury or wound needs to be stopped urgently, a rubber band or string is used to wrap the finger tightly. While this method is effective to stop bleeding immediately, it gives rise to a risk of severe irreversible ischemic tissue damage due to complete occlusion of both digital arteries and veins. For example, if the finger is tied up by the rubber band for more than 20 minutes, irreversible tissue necrosis can occur, requiring tragic finger amputation. This incident was reported in medical journals.

### Advantages

The advantages of the present finger tip tourniquet include:
1. Much less puncture pain from the ability to use much smaller and thinner lancet needles.
2. Less puncture injury and faster puncture wound healing from the use of smaller and thinner needles.
3. Economical gain from less waste of diagnostic test strips. If insufficient blood amount was produced by the finger stick when this finger tourniquet is not used, the test strip will be wasted.
4. Improved patient's comfort because of the selective compression of the part of the finger (ventral and dorsal area only) by the compression pads having soft cushion linings.
5. Little risk of serious irreversible tissue damage from the prolonged application such as a forgotten finger tourniquet.
6. When this finger tourniquet is applied to one or both lateral sides of the finger, it restricts mainly the digital arterial blood influx while allowing venous blood drainage. While significant bleeding can be stopped, because of the small amount of blood still flowing into the finger tip through the smaller branches of the digital artery located in elsewhere other than the lateral sides of the finger with the open venous draining system, irreversible ischemic tissue damage will not occur even with longer application time. This method is very useful as the first aid for a finger injury by a layman before obtaining professional medical care.

Although the present invention has been discussed in considerable detail with reference to certain preferred embodiments, other embodiments are possible. The steps disclosed for the present methods are not intended to be limiting nor are they intended to indicate that each step is necessarily essential to the method, but instead are exemplary steps only. Therefore, the scope of the appended claims should not be limited to the description of preferred embodiments contained in this disclosure. All references cited herein are incorporated by reference in their entirety.

What is claimed is:

1. A tourniquet for compressing a finger having a ventral surface and a dorsal surface, comprising:
a first compression pad comprising a first proximal side, a first distal side and a first surface between the first proximal side and the first distal side;
a second compression pad comprising a second proximal side, a second distal side, and a second surface between the second proximal side and the second distal side, wherein the first surface of the first compression pad faces the second surface of the second compression pad and is separated from the second surface by a distance; and
means for tensioning the first and second compression pads when the distance between the first surface of the first compression pad and the second surface of the second compression pad is increased.

2. The tourniquet of claim 1, wherein the means for tensioning comprises a spring connector.

3. The tourniquet of claim 2, wherein the means for tensioning comprises a leaf spring.

4. The tourniquet of claim 2, further comprising a first handle connected to the first compression pad and a second handle connected to the second compression pad for tensioning the spring connector.

5. The tourniquet of claim 2, wherein a first end of the spring connector is attached to the first handle and a second end of the spring connector is attached to the second handle.

6. The tourniquet of claim 2, further comprising means for adjusting the tension of the spring connector.

7. The tourniquet of claim 1, wherein the means for tensioning the first and second compression pads comprises elastic material connected to the first and second compression pads, wherein the elastic material connects the first proximal side of the first compression pad to the second proximal side of the second compression pad, and wherein the elastic material further connects the first distal side of the first compression pad to the second distal side of the second compression pad.

8. The tourniquet of claim 7, wherein the first surface and the second surface are concave.

9. The tourniquet of claim 7, wherein the distance between the proximal side and the distal side of the first compression pad and the distance between the proximal side and the distal side of the second compression pad is greater than the width of the finger.

10. The tourniquet of claim 9, wherein the distance between the proximal side and the distal side of the first compression pad and the distance between the proximal side and the distal side of the second compression pad is at least 2.0 centimeters.

11. The tourniquet of claim 10, wherein when the elastic material is not under tension, the distance between the first proximal side of the first compression pad and the second proximal side of the second compression pad, and the distance between the first distal side of the first compression pad and the second distal side of the second compression pad, is 0.5 centimeters or less.

12. A method of obtaining a blood sample from a distal end of a finger, the finger having a ventral surface and a dorsal surface, comprising:
(a) providing the tourniquet of claim 1;
(b) tensioning the means for tensioning the first and second compression pads, thereby increasing the distance between the first surface of the first compression pad and the second surface of the second compression pad;
(c) inserting the distal end of the finger between the first and second compression pads of the tourniquet;
(d) positioning the first surface of the first compression pad adjacent dorsal surface of the finger and positioning the second surface of the second compression pad adjacent the ventral surface of the finger;
(e) releasing tension in the means for tensioning the first and second compression pads, thereby decreasing the distance between the first surface of the first compression pad and the second surface of the second compression pad and compressing the ventral and dorsal surfaces of the finger; and
(f) puncturing skin of the finger with a lancet in order to obtain the blood sample.

13. The method of claim 12, wherein:
the distance between the proximal side and the distal side of the first compression pad and the distance between the proximal side and the distal side of the second compression pad is greater than the width of the finger,
when the elastic material is not under tension, the distance between the first proximal side of the first compression pad and the second proximal side of the second compression pad, and the distance between the first distal side of the first compression pad and the second distal side of the second compression pad is less than the thickness of the finger, and
the first surface of the first compression pad and the second surface of the second compression pad are placed in contact with the dorsal and ventral surfaces of the finger, respectively.

14. A method of reducing arterial blood flow to a distal end of a finger, the finger having a pair of lateral sides between a ventral surface and a dorsal surface of the finger, comprising:
(a) providing the tourniquet of claim 1;
(b) tensioning the means for tensioning the first and second compression pads, thereby increasing the distance between the first surface of the first compression pad and the second surface of the second compression pad;
(c) inserting the distal end of the finger between the first and second compression pads of the tourniquet
(d) positioning the first surface of the first compression pad adjacent one of the lateral sides of the finger and positioning the second surface of the second compression pad adjacent the other lateral side;
(e) releasing tension in the means for tensioning the first and second compression pads, thereby decreasing the distance between the first surface of the first compression pad and the second surface of the second compression pad, compressing the lateral sides of the finger, and reducing arterial blood flow to the distal end of the finger.

15. A method of obtaining a blood sample from a distal end of a finger, the finger having a pair of lateral sides between a ventral surface and a dorsal surface of the finger, the lateral sides being separated by a distance, comprising:
(a) providing a tourniquet for compressing the lateral sides of the finger, comprising:
(i) a rigid compression pad comprising a first medial side, a second medial side and a first surface between the first medial side and the second medial side, wherein the distance between the first medial side and the second medial side is greater than the distance between the lateral sides of the finger; and
(ii) an elastic material having a first end and a second end, wherein the first end is connected to the first medial side of the rigid compression pad and the second end is connected to the second medial side of the rigid compression pad, the first surface of the rigid compression pad facing the inner surface of the elastic material;
(b) inserting a distal end of the finger between the first surface of the rigid compression pad and the elastic material;
(c) positioning the first surface of the rigid compression pad on the dorsal surface of the finger.
